(19) United States
(12) Patent Application Publication
(10) Pub. No.: US 2025/0228875 A1
(43) Pub. Date: Jul. 17, 2025

(54) METHODS OF TREATING, AMELIORATING AND/OR PREVENTING FIBRODYSPLASIA OSSIFICANS PROGRESSIVA AND HETEROTOPIC OSSIFICATION, AND KITS FOR THE SAME

(71) Applicant: The Trustees of the University of Pennsylvania, Philadelphia, PA (US)

(72) Inventors: Frederick S. Kaplan, Philadelphia, PA (US); Robert J. Pignolo, Rochester, MN (US); Vitali Lounev, Wynnewood, PA (US)

(21) Appl. No.: 18/852,841

(22) PCT Filed: Mar. 28, 2023
(86) PCT No.: PCT/US2023/065067

§ 371 Date: Sep. 30, 2024

Related U.S. Application Data
(60) Provisional application No. 63/324,842, filed on Mar. 29, 2022.

(51) Int. Cl. A61K 31/65; A61K 31/7088; A61K 38/46; A61K 38/48; A61K 48/00; C12N 9/22; C12N 15/11; C12N 15/113

(52) U.S. Cl. CPC A61K 31/65; A61K 31/7088; A61K 38/465; A61K 38/4886; A61K 48/005; C12N 9/22; C12N 15/11; C12N 15/1137

(57) ABSTRACT

Disclosed herein are methods for treating, ameliorating, and/or preventing fibrodysplasia ossificans progressiva (FOP) in a subject in need thereof. The methods include downregulating matrix metalloproteinase 9 (MMP-9) level and/or activity in the subject. Also disclosed are kits for carrying out the methods disclosed herein.

### CROSS-REFERENCE TO RELATED APPLICATIONS

The present application claims priority under 35 U.S.C. § 119(e) to U.S. Provisional Patent Application No. 63/324,842, filed Mar. 29, 2022, which is incorporated herein by reference in its entirety.

### REFERENCE TO SEQUENCE LISTING

The Sequence Listing concurrently submitted herewith as an xml file named “046483-7360WO1.xml,” created on Mar. 28, 2023 and having a size of 4,200 bytes is herein incorporated by reference.

### BACKGROUND

Fibrodysplasia ossificans progressiva (FOP; MIM #135100) is the most catastrophic form of extraskeletal bone formation in humans. In this disease, muscle tissue and connective tissue such as tendons and ligaments are gradually replaced by bone (ossified), forming bone outside the skeleton (extra-skeletal or heterotopic bone) that constrains movement.

Individuals with FOP appear normal at birth except for characteristic malformations of the great toes that are present in all classically affected individuals. During the first decade of life, episodic soft tissue swellings (or flare-ups) arise in the neck and back and undergo pathological metamorphosis into mature heterotopic bone through an endochondral pathway. Minor trauma such as intramuscular immunizations, mandibular blocks for dental work, muscle over-exertion, blunt muscle trauma, bumps, bruises, falls, or influenza-like viral illnesses can trigger flare-ups of FOP that lead to progressive heterotopic ossification (HO). Most patients are immobilized by the age 30, and require lifelong assistance with activities of daily living. The median estimated lifespan is 56 years; death often results from complications of thoracic insufficiency syndrome.

Definitive treatments are not yet available for FOP, and the current standard-of-care medical management is only supportive. There is a need for compositions and methods that can be used for treating, ameliorating, and/or preventing FOP. The present invention addresses this need.

### SUMMARY

In some aspects, the present invention is directed to the following non-limiting embodiments:

In some aspects, the present invention provides a method of treating, ameliorating, and/or preventing fibrodysplasia ossificans progressiva (FOP) in a subject in need thereof.

In some embodiments, the method comprises down-regulating matrix metalloproteinase 9 (MMP-9) level and/or activity in the subject.

In some embodiments, down-regulating the MMP-9 level and/or activity in the subject comprises administering to the subject an effective amount of:
a small molecule MMP-9 inhibitor,
a protein MMP-9 inhibitor,
a nucleic acid (and/or an expression vector expressing the nucleic acid) that downregulates MMP-9 by RNA interference,
a ribozyme (and/or a vector expressing the ribozyme) that downregulates MMP-9,
an expression vector including an expression cassette, wherein the expression cassette expresses CRISPR components that downregulate MMP-9 by CRISPR knockout and/or CRISPR knockdown, and
a trans-dominant negative mutant protein of MMP-9, and/or an expression vector that expresses the trans-dominant negative mutant protein of MMP-9.

In some embodiments, the subject has a mutant ACVR1 gene.

In some embodiments, the mutant ACVR1 gene encodes a constitutively active ACVR1 polypeptide.

In some embodiments, the ACVR1 polypeptide comprises at least one mutation selected from the group consisting of L196P, P197-F198 del ins L, R202I, R206H, Q207E, F246Y, R258S, R258G, G325A, G328E, G328W, G328R, G356D, R375P, and K400E.

In some embodiments, the the small molecule MMP-9 inhibitor is selected from the group consisting of doxycycline, incyclinide, and minocycline, or a salt or solvate thereof.

In some embodiments, the protein MMP-9 inhibitor is an anti-MMP-9 antibody or an antigen binding fragment thereof.

In some embodiments, the method further comprises surgically removing an ossified tissue from the subject.

In some embodiments, the surgically removing step is performed after the MMP-9 level or activity in the subject is down-regulated.

In some embodiments, the subject is a human.

In some aspects, the present invention is directed to a kit for treating, ameliorating and/or preventing fibrodysplasia ossificans progressiva (FOP) in a subject in need thereof.

In some embodiments, the kit comprises: a compound for down-regulating matrix metalloproteinase 9 (MMP-9) level and/or activity in the subject; and an instruction for administering an effective amount of the compound to the subject.

In some embodiments, the compound comprises at least one selected from the group consisting of:
a small molecule MMP-9 inhibitor,
a protein MMP-9 inhibitor,
a nucleic acid (and/or an expression vector expressing the nucleic acid) that downregulates MMP-9 by RNA interference,
a ribozyme (and/or a vector expressing the ribozyme) that downregulates MMP-9,
an expression vector including an expression cassette, wherein the expression cassette expresses CRISPR components that downregulate MMP-9 by CRISPR knockout and/or CRISPR knockdown, and
a trans-dominant negative mutant protein of MMP-9, and/or an expression vector that expresses the trans-dominant negative mutant protein of MMP-9.

In some embodiments, the subject has a mutant ACVR1 gene.

In some embodiments, the mutant ACVR1 gene encodes a constitutively active ACVR1 polypeptide.

In some embodiments, the ACVR1 polypeptide comprises at least one mutation selected from the group consisting of L196P, P197-F198 del ins L, R202I, R206H, Q207E, F246Y, R258S, R258G, G325A, G328E, G328W, G328R, G356D, R375P, and K400E.

In some embodiments, the small molecule MMP-9 inhibitor is selected from the group consisting of doxycycline, incyclinide, and minocycline.

In some embodiments, the protein MMP-9 inhibitor is an antibody against MMP-9 or an antigen binding fragment thereof.

In some embodiments, the instruction further comprises instructions for surgically removing an ossified tissue from the subject.

In some embodiments, the instruction further comprises instructions to perform the surgically removing after the level or the activity of MMP-9 in the subject is down-regulated.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 shows the progression of FOP in a typical patient, in accordance with some embodiments.

FIGS. 2A-2D show the characterizations of FOP in Patient-R, in accordance with some embodiments. FIG. 2A is the photo of malformations in toes, FIG. 2B is the radiographs of toes, and FIG. 2C is the scout CT image (collage), which shows the presence of congenital features of FOP and paucity of HO in patient-R. FIG. 2D is an electropherogram showing the classic ACVR1 mutation (R206H, 617G>A) in patient-R.

FIGS. 3A-3C show the results of MMP-9 assays in Patient-R, in accordance with some embodiments. FIG. 3A shows that Patient-R has lower plasma MMP-9 (Myriad RBM assay) levels than all other FOP patients and controls (**p<0.01, n=77). FIG. 3B is the results of gelatin zymography assays which reveal less total MMP-9 in PBMCs of patient-R. FIG. 3C depicts MMP-9 enzymatic activity of PBMCs from Patient-R versus those from control subjects and other FOP patients. The results show decreased reserve of MMP-9 in PBMCs from Patient-R compared to PBMCs from other FOP patients.

FIGS. 4A-4B shows the evaluation of MMP-9 polymorphisms in Patient-R, in accordance with some embodiments. FIG. 4A: Cartoon of MMP-9 protein denoting the various anatomic locations of the A20V and D165N SNPs resulting in amino acid residue changes. FIG. 4B: Sequence of PCR amplicons confirms the presence of A20V and D165N SNPs in Patient-R.

FIG. 5 demonstrates that MMP-9 is expressed in early lesional tissues in accordance with some embodiments. FIG. 5 shows images of early lesional tissue of FOP model mice (Acvr1^{R206H/+};CreERT2^{-/+}) at 1, 3 and 5 days after cardiotoxin injury, which show MMP-9 expression in inflammatory cells.

FIGS. 6A-6N demonstrate that down-regulating MMP-9 in FOP mouse models with genetic, pharmacological or biological means all resulted in decreased HO. FIG. 6A: both MMP9^{-/+};Acvr1^{R206H/+};CreERT2^{-/+} mice and MMP9^{-/-};Acvr1^{R206H/+};CreERT2^{-/+} mice form significantly less HO than Acvr1R^{206H/-};CreERT2^{-/+} mice following soft tissue injury. FIG. 6B depicts the quantitation of HO bone volumes in the experiment of FIG. 6A, as analyzed by MicroCT (***p<0.001, n=6). FIG. 6C: MMP9^{-/+};Acvr1^{R206H/+};CreERT2^{-/+} and MMP9^{-/-};Acvr1^{R206H/+};CreERT2^{-/-} mice produce significantly less MMP-9 than Acvr1^{R206H/+;CreERT2-/+} control mice. FIG. 6D shows the histology (H&E staining) of MMP9^{+/+};Acvr1^{+/+}, MMP9^{+/+};Acvr1^{R206H/+};CreERT2^{-/+}, MMP9^{-/-};Acvr1^{+/+}, and MMP9^{-/-};Acvr1^{R206H/+};CreERT2^{-/+} mice after injury with cardiotoxin at 14 days. FIG. 6E: Acvr1^{R206H/+};CreERT2^{-/+} mice treated with minocycline form significantly less HO than Acvr1^{R206H/+};CreERT2^{-/+} control mice following soft tissue injury. FIG. 6F depicts the quantitation of HO bone volumes in the experiment of FIG. 6E as analyzed by MicroCT (**p<0.01, n=6). FIG. 6G: Acvr1^{Q207D/+} mice treated with minocycline form significantly less HO than untreated Acvr1^{Q207D/+} mice following soft tissue injury (***p<0.001, n=6). FIG. 6H depicts the quantitation of HO bone volumes of the experiment of FIG. 6G as analyzed by MicroCT. FIG. 6I shows that minocycline protects against HO when administered before or at the time of lesional activation, indicating that MMP-9 acts to induce HO during the early inflammatory stage. FIG. 6J shows the effect of minocycline at different concentrations on HO in Acvr1^{Q207D/+} mice. Minocycline at a dose of 5 to 100 mg/kg is effective in decreasing HO. FIG. 6K depicts the results of blocking mAbMMP-9 (Gilead) on HO bone volume in Acvr1^{R206H/+};CreERT2^{-/+} FOP mice following soft tissue injury. FIG. 6L is the result of gelatin zymography assay. Cell culture supernatants were from M1-like and M2-like macrophages at 48 hours after polarization of THP-1, THP-1^{A20V}, and THP-1D^{165N} M0 macrophages. FIG. 6M shows quantification of MMP-9 enzymatic activity in gelatin zymography. MMP-9^{D165N} enzymatic activity is reduced compared to MMP-9^{WT} and MMP-9^{A20V} in M1-like and M2-like macrophages. FIG. 6N shows the ELISA for Activin A in cell culture supernatants from M1-like and M2-like macrophages at 48 hours after polarization of THP-1, THP-1^{A20V} and THP-1^{D165N} M0 macrophages. The level of Activin A in the supernatant is dramatically reduced by MMP-9^{A20V} and MMP-9^{D165N} compared to MMP-9^{WT}.

FIG. 7 illustrates a proposed molecular mechanism of MMP-9 involvement in FOP, in accordance with some embodiments.

FIG. 8 shows protein-protein interaction (PPI) mapping result of MMP-9 connectivity in accordance with some embodiments. The PPI mapping was performed with IBM-Watson for Drug Discovery (WDD) and Ingenuity software.

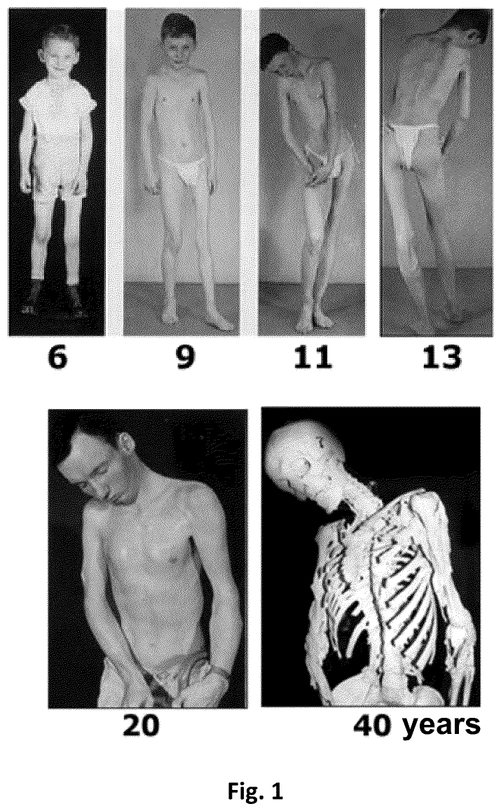

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Heterozygous missense mutations in activin receptor A type I, a bone morphogenetic protein (BMP) type I receptor, have been identified in all individuals with sporadic or familial fibrodysplasia ossificans progressiva (FOP). ACVR1 mutations cause loss of autoinhibition of ACVR1 and render it susceptible to dysregulated BMP pathway signaling. Activin A, a member of the transforming growth factor-β (TGF-β) family of molecules that antagonizes BMP signaling in a wild-type (WT) ACVR1 background, specifically enhances BMP pathway signaling within cells harboring the ACVR1R206H mutation and drives heterotopic bone formation in FOP.

The study described herein (“the present study”) discovered a special fibrodysplasia ossificans progressiva (FOP) patient having the classical R206H mutation in Acvr1 BMP receptor. Although this patient had the congenital features of FOP, he developed very few post-natal FOP features. The present study discovered that this patient had significantly suppressed biomarkers of inflammation, and that the patient had a compound heterozygosity for MMP-9 gene (one allele of the MMP-9 gene having a polymorphism resulting in an A20V mutation in the expressed polypeptide and the other allele having a polymorphism resulting in a D165N mutation). Using protein structure modeling, the present study predicted that the compound heterozygosity of MMP-9 found in the patient resulted in reduced MMP-9 level and activity in the patient. The present study discovered that MMP-9 is expressed in early lesional tissues of FOP subjects. Using several mouse FOP models, the present study discovered that reducing MMP-9 level and/or activity by genetic deletion of MMP-9 (including partial deletion), administration of non-limiting examples of monoclonal antibodies against MMP-9, or administration of non-limiting small molecule inhibitors of MMP-9 all resulted in decreased level of heterotopic ossification (bone formation) in response to injury in the mouse FOP models.

Accordingly, in some aspects, the instant invention is directed to a method of treating, ameliorating, and/or preventing fibrodysplasia ossificans progressiva (FOP) in a subject in need thereof.

In some aspects, the instant invention is directed to a kit for treating, ameliorating, and/or preventing FOP in a subject in need thereof.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments 5%, in certain embodiments ±10%, in certain embodiments ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

The terms “fibrodysplasia ossificans progressiva” or “FOP,” as used herein, refer to a genetic disorder of congenital skeletal malformations and progressive heterotopic ossification (HO). Heterozygous (i.e., patients have one copy of mutant Acvr1 gene and one copy of wildtype Acvr1 gene) missense mutations in activin receptor A type I/Activin-like kinase 2 (ACVR1/ALK2), a bone morphogenetic protein (BMP) type I receptor, have been identified in all individuals with sporadic or familial FOP. An activating R206H mutation on ACVR1 is found in most FOP patients. Details of this disease is described in, for example, Kaplan et al. (Best Practice & Research Clinical Rheumatology Volume 22, Issue 1, March 2008, Pages 191-205) and Kaplan et al. (Bone Volume 140, November 2020, 115539).

The terms “heterotopic ossification” or “HO,” as used herein, refer to the formation of extraskeletal bone in muscle and soft tissues. HO is one of the post-natal features of FOP, and is a common occurrence after trauma in non-FOP patients. Details of heterotopic ossification is described in, for example, Meyers et al. (JBMR Plus 2019, 3: e10172) and Dey et al., (Transl Res. 2017 August; 186: 95-111).

The term “flare-up,” when used in connection to FOP herein, are inflammatory soft tissue swellings experienced by most FOP patients, which begin during early childhood and progress throughout life. Flare-ups include unpredictable episodes of soft tissue swelling, pain, reduced movement, and/or stiffness. These flare-ups usually result in extra bone formation, but not always. Details of flare-ups in FOP is described in, for example, Pignolo et al. (J Bone Miner Res. 2016 March;31(3):650-6).

Non-limiting abbreviations used herein: FOP: fibrodysplasia ossificans progressiva. HO: heterotopic ossification. PBMC: peripheral blood mononuclear cell. SNP: single nucleotide polymorphism.

Method of Treating, Ameliorating and/or Preventing Fibrodysplasia Ossificans Progressiva

The present study discovered a fibrodysplasia ossificans progressiva (FOP) patient having the classical R206H mutation in Acvr1 BMP receptor. Although this patient had the congenital features of FOP, developed very few post-natal FOP features. The present study discovered that this patient had significantly suppressed biomarkers of inflammation, and that the patient had a compound heterozygosity for MMP-9 gene (one allele of the MMP-9 gene having a polymorphism resulting in A20V and the other allele having a polymorphism resulting in D165N). Using protein structure modeling, the present study predicted that the compound heterozygosity of MMP-9 found in the patient resulted in reduced MMP-9 level and activity in the patient. The present study discovered that MMP-9 is expressed in early lesional tissues of FOP subjects. Using several mouse FOP models as non-limiting examples, the present study discovered that reducing MMP-9 level and/or activity by genetic deletion of MMP-9 (including partial deletion), administration of a non-limiting MMP-9 monoclonal antibody, or administration of a non-limiting small molecule inhibitor of MMP-9 all resulted in decreased level of heterotopic ossification (bone formation) in response to soft tissue injury.

Accordingly, in some aspects, the present invention is directed to a method of treating, ameliorating and/or preventing fibrodysplasia ossificans progressiva (FOP) in a subject. In some embodiments, the method includes administering to the subject a compound that down-regulates MMP-9 level and/or activity in the subject.

The present study discovered that the inhibition of MMP-9 is able to significantly reduce the level of heterotopic ossification in model animals with Q270D mutation in Acvr1 BMP receptor. Since Q270D mutation does not cause spontaneous heterotopic ossification but rather results in HO in response to injury only and produces very robust post-traumatic HO, the Q270D model is sometimes considered a model for HO. Accordingly, In some aspects, the present invention is directed to a method of treating, ameliorating and/or preventing heterotopic ossification in a subject in need thereof. In some embodiments, the method includes administering to the subject a compound that down-regulates MMP-9 level and/or activity in the subject.

In some embodiments, the subject is treated with a bone marrow transplantation which replaces some hematopoietic stem cells in the subject with those that having reduced level and/or activity of MMP-9. One of ordinary skill in the art would understand that MMP-9, although expressed in multiple tissues, is strongly expressed in bone marrow and lymphoid tissues. As described elsewhere herein, the present study discovered that partial reduction of MMP-9 expression by deleting only one MMP-9 allele is sufficient to counter the soft tissue injury induced heterotopic ossification in FOP model animals. As such, in certain embodiments, partial reduction and/or elimination of MMP-9 expression and/or activity only in the bone marrow is sufficient to mimic the partial reduction of MMP-9 expression in FOP model mice. In some embodiments, the bone marrow stem cells used for the transplantation are genetically engineered to reduce MMP-9 level and/or activity. Genetic engineering of the hematopoietic stem cells in the bone marrow is described in, for example, Daniel-Moreno et al. (Bone Marrow Transplantation volume 54, pages 1940-1950 (2019)). In some embodiments, the bone marrow used for the transplantation is from a donor having MMP-9 polymorphisms that reduce the MMP-9 level and/or activity, such as the MMP-9 polymorphisms found in Patient-R described herein.

In some embodiments, the subject has a mutant ACVR1 gene. In some embodiments, the mutant ACVR1 gene encodes a constitutively active ACVR1 polypeptide. In other embodiments, the mutant ACVR1 gene comprises, relative to a wild-type gene, a mutation (e.g., one or more point mutations, deletions, and/or insertions) in the nucleic acid sequence of the ACVR1 gene encoding the ACVR1 polypeptide. In some embodiments, the ACVR1 polypeptide comprises a mutation. In some embodiments, the ACVR1 polypeptide comprises one or more amino acid substitutions, deletions, and/or insertions relative to a wild-type polypeptide. In some embodiments, the ACVR1 polypeptide comprises one or more amino acid substitutions, deletions, and/or insertions relative to a wild-type ACVR1 polypeptide, wherein the wild-type ACVR1 polypeptide comprises the amino acid sequence set forth in SEQ ID NO:3. Genbank Accession No. NP_001104537, which is herein incorporated by reference in its entirety, discloses an ACVR1 polypeptide. In some embodiments, the ACVR1 polypeptide comprises one or more mutations on amino acid residues L196, P197, F198, R202, R206, Q207, F246, R258, G325, G328, G356, R375 or K400. In some embodiments, the ACVR1 polypeptide comprises one or more mutations on amino acid residues L196, P197, F198, R202, R206, Q207, F246, R258, G325, G328, G356, R375 or K400 relative to the wild-type ACVR1 polypeptide comprising the sequence set forth in SEQ ID NO:3. In some embodiments, the ACVR1 polypeptide comprises one or more mutations selected from the group consisting of L196P, P197-F198 del ins L, R202I, R206H, Q207E, F246Y, R258S, R258G, G325A, G328E, G328W, G328R, G356D, R375P, and K400E, wherein del represents deletion and ins represents insertion. In some embodiments, the ACVR1 polypeptide comprises one or more mutations selected from the group consisting of L196P, P197-F198 del ins L, R202I, R206H, Q207E, F246Y, R258S, R258G, G325A, G328E, G328W, G328R, G356D, R375P, and K400E relative to the wild-type ACVR1 polypeptide comprising the sequence set forth in SEQ ID NO:3, wherein del represents deletion and ins represents insertion. ACVR1 mutations involved in FOP are also described in Haupt et al. (Bone Volume 109, April 2018, Pages 232-240) and Mukaddam et al., (Genetics of Fibrodysplasia Ossificans Progressiva, eLS, Vol 2: 1-8, 2021), each of which is herein incorporated by reference in its entirety.

In some embodiments, the method further comprises surgically removing an ossified tissue from the subject. In some embodiments, the surgical removal of ossified tissue is performed after the level or the activity of MMP-9 in the subject has been down-regulated.

In some embodiments, the subject is a mammal. In some embodiments, the subject is a human.

### Small Molecule Inhibitor of MMP-9

In some embodiments, the compound that down-regulates MMP-9 level and/or activity includes a small molecule inhibitor of MMP-9.

As used herein, the term “small molecule” means molecules have a molecular weight of about 2000 daltons or less, such as about 1800 daltons or less, about 1600 daltons or less, about 1400 daltons or less, about 1200 daltons or less, about 1000 daltons or less, or about 900 daltons or less. Non-limiting examples of small molecules that inhibit MMP-9 include actinonin, ageladine A TFA, apigenin-7-glucuronide, ARP 100, astragaloside IV, BR351, chlorhexidine dihydrochloride, cipemastat, CMC2.24, CP-471474, CP-544439, cyclic CTTHWGFTLC, cyclic CTTHWGFTLC TFA, FSL-1 TFA, ginkgolide C, ilomastat (also referred to as GM6001), JNJ0966, luteolin 7-O-glucuronide, marimastat, MMP-2/MMP-9 Inhibitor I, MMP-2/MVP-9 Inhibitor II, MMP3 inhibitor 1, MMP-9-IN-1, MMP-9 Inhibitor I, MMP-9 Inhibitor II, MMP-9/MMP-13 inhibitor I, MMP Inhibitor II, MMP13-IN-3, MMPI-1154, morroniside, ND-336, NNGH, (R)-ND-336, PF-00356231 hydrochloride, PD-166793, prinomastat, prinomastat hydrochloride, salvianolic acid A, S 3304, SB-3CT, SM-7368, tanomastat, tetracycline derivatives such as doxycycline, incyclinide, and minocycline, UK 356618, UK-370106, XL-784, and the like. MMP-9 inhibitors are described in, for example, Fields (Cells. 2019 September; 8(9): 984.)

In some embodiments, the small molecule inhibitor of MMP-9 includes a selective MMP-9 small molecule inhibitor. As used herein, the term “selective MMP-9 inhibitor” refers to small molecule compounds that having IC₅₀ toward MMP-9 equal to or higher than the IC₅₀ toward any other matrix metalloproteinases, such as higher by 0.1 or more, higher by 0.2 or more, higher by 0.3 or more, higher by 0.5 or more, higher by 0.8 or more, or higher by 1.0 or more than the IC₅₀ toward any other matrix metalloproteinases. Non-limiting examples of selective MMP-9 small molecule inhibitors include apigenin-7-glucuronide, FFAGLDD, FSL-1 TFA, ginkgolide C, isoliquiritin apioside, JNJ0966, luteolin 7-O-glucuronide, MMP-9-IN-1, (R)-ND-336, SM-7368, FFAGLDD TFA, and the like.

In some embodiments, the small molecule inhibitor of MMP-9 includes a compound that are in use in the medical field for purposes other than inhibiting MMP-9 and is known to be generally safe. Non-limiting examples of such compounds include tetracyclines such as doxycycline, incyclinide, and minocycline. Examples of tetracyclines that are able to inhibit MMP-9 are described in, e.g., Griffin et al. (Am J Physiol Cell Physiol. 2010 September; 299(3): C539-C548.), the entirety of the reference is hereby incorporated herein by reference.

### Protein Inhibitor of MMP-9

In some embodiments, the compound that down-regulates MMP-9 level and/or activity includes a protein inhibitor of MMP-9.

In some embodiments, the protein inhibitor of MMP-9 includes an antibody against MMP-9, or an antigen binding fragment thereof. Examples of antibody against MMP-9 include anti-Ac-MMP-9 antibody (4A3), anti-MMP-9 antibody (E-11), anti-MMP-9 antibody (2C3), and anti-MMP-9 antibody (6-6B) by Santa Cruz Biotechnology, MMP9 monoclonal antibody (5G3), MMP9 Recombinant Rabbit Monoclonal Antibody (JA80-73), and MMP9 Monoclonal Antibody (5C3) by Invitrogen, and the like.

In some embodiments, the protein inhibitor of MMP-9 includes non-antibody protein or peptide inhibitor of MMP-9. Non-limiting examples of non-antibody protein or peptide inhibitors of MMP-9 include proteins from the tissue inhibitors of metalloproteinase (TIMP) family such as TIMP-1, TIMP-3 and the like, FFAGLDD peptide, FFAGLDD TFA, cyclic CTTHWGFTLC, cyclic CTTHWGFTLC TFA, and the like.

### RNA Interference Nucleic Acids for Downregulate MMP-9 and Vectors Expressing the Same

In some embodiments, the compound that down-regulates MMP-9 level and/or activity includes a nucleic acid that downregulates MMP-9 level by RNA interference, and/or an expression vector expressing the nucleic acid.

In some embodiments, the nucleic acid that downregulates the level of MMP-9 by the means of RNA interference includes an isolated nucleic acid. In other embodiments, the modulator is an RNAi molecule (such as but not limited to siRNA and/or shRNA and/or miRNAs) or antisense molecule, which inhibits MMP-9 expression and/or activity. In yet other embodiments, the nucleic acid comprises a promoter/regulatory sequence, such that the nucleic acid is preferably capable of directing expression of the nucleic acid. Thus, the instant specification provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of MMP-9. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; Engelke, Ed., RNA Interference (RNAi) Nuts & Bolts of RNAi Technology, DNA Press, Eagleville, PA (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the instant specification also includes methods of decreasing levels of MMP-9 using RNAi technology.

In certain embodiments, the instant specification provides a vector comprising an siRNA or antisense polynucleotide. In other embodiments, the siRNA or antisense polynucleotide inhibits the expression of MMP-9. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

To assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide has certain characteristics that can be modified to improve the siRNA as a therapeutic compound. Therefore, in some embodiments, the siRNA polynucleotide is further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

In certain embodiments, an antisense nucleic acid sequence expressed by a plasmid vector is used to inhibit MMP-9 protein expression. The antisense expressing vector is used to transfect a mammalian cell or the mammal itself, thereby causing reduced endogenous expression of MMP-9.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the instant specification may be made synthetically and then provided to the cell. Antisense oligomers of between about 10 to about 30, and more preferably about 15 nucleotides, can be used, since they are easily synthesized and introduced into a target cell. Synthetic antisense molecules contemplated by the instant specification include oligonucleotide derivatives known in the art which have improved biological activity compared to unmodified oligonucleotides (see U.S. Pat. No. 5,023,243).

Ribozyme that Downregulates AMP-9 and Vectors Expressing the Same

In some embodiments the compound that down-regulates MMP-9 level and/or activity includes a ribozyme that downregulates MMP-9, and/or a vector expressing the ribozyme.

A ribozyme is used to inhibit MMP-9 protein expression. Ribozymes useful for inhibiting the expression of a target molecule may be designed by incorporating target sequences into the basic ribozyme structure which are complementary, for example, to the mRNA sequence encoding MMP-9. Ribozymes are antisense RNAs which have a catalytic site capable of specifically cleaving complementary RNAs. Therefore, ribozymes having sequence complementary to MMP-9mRNA sequences can downregulate the expression of MMP-9 by reduces the level of MMP-9 mRNA. Ribozymes targeting MMP-9, may be synthesized using commercially available reagents (Applied Biosystems, Inc., Foster City, CA) or they may be genetically expressed from DNA encoding them. In some embodiments, the DNA encoding the ribozymes are incorporated in a vector described elsewhere herein.

Compounds that Downregulate MMP-9 by CRISPR Knockout/Knockdown and Other Knockout Knockdown Techniques

In some embodiments the compound that down-regulates MMP-9 level and/or activity includes a compound that downregulates MMP-9 by CRISPR knockout/knockdown and other knockout/knockdown techniques. In some embodiments, the compound includes an expression vector including an expression cassette, wherein the expression cassette expresses CRISPR components that downregulate MMP-9 by CRISPR knockout or CRISPR knockdown.

In some embodiments, the compound that down regulates the activity or level of MMP-9 comprises a CRISPR/Cas9 system for knocking out MMP-9.

The CRISPR/Cas9 system is a facile and efficient system for inducing targeted genetic alterations. Target recognition by the Cas9 protein requires a “seed” sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to neurons, stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the instant specification. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the instant specification may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, the compound that down regulates the activity or expression level of MMP-9 comprises a nucleic acid that down regulates the expression level of MMP-9 by the means of CRISPR knockdown. CRISPR knockdown includes, but not limited to, CRISPRCas13 knockdown. (See e.g., Mendez-Mancilla et al., Cell Chemical Biology 29, 1-7, 2021 Jul. 27, and Kushawah et al., Dev Cell. 2020 Sep. 28; 54(6):805-817. The entireties of which are incorporated herein by reference).

In some embodiments, the present invention includes any other methods for effecting gene knockdown and/editing, which allow for deletion and/or inactivation of MMP-9, such as but not limited to those described in WO 2018/236840 (which is incorporated herein in its entirety by reference).

Compounds that Downregulate MMP-9 by Inactivating and or Sequestering

In some embodiments, the compound that downregulates the activity or expression level of MMP-9 includes a protein that downregulates the activity of MMP-9 by inactivating and/or sequestering MMP-9. In some embodiment, the compound includes a nucleic acid that express the protein that downregulates the activity of MMP-9 by inactivating and/or sequestering MMP-9. In some embodiments, the compound includes an expression vector that express the protein that downregulates the activity of MMP-9 by inactivating and/or sequestering MMP-9 (see “Vector” section for non-limiting descriptions on vectors).

In some embodiments, the compound that downregulates the expression level of MMP-9 is a trans-dominant negative mutant of MMP-9, and/or a nucleic acid or a vector expressing the trans-dominant negative mutant of MMP-9.

Kit for Treating, Ameliorating and/or Preventing Fibrodysplasia Ossificans Progressiva

In some aspects, the present invention is directed to a kit for treating, ameliorating and/or preventing fibrodysplasia ossificans progressiva (FOP) in a subject in need thereof.

In some aspects, the present invention is directed to a kit for treating, ameliorating and/or preventing heterotopic ossification in a subject in need thereof.

In some embodiments, the kit includes a compound that down-regulates MMP-9 level and/or activity in the subject; and a manual instructing that an effective amount of the compound be administered to the subject. In some embodiments, the compound that down-regulates MMP-9 level and/or activity in the subject is the same as or similar to those as described elsewhere herein, such as in the “Method of Treating, Ameliorating and/or Preventing Fibrodysplasia Ossificans Progressiva” section.

### Vectors

Vectors can increase the stability of the nucleic acids, make the delivery easier, or allow the expression of the nucleic acids or protein products thereof in the cells.

Therefore, in some embodiments, the protein inhibitors or the nucleic acids that that down regulates the activity or expression level of MMP-9 is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the nucleic acids that downregulates MMP-9 described above, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the bone marrow, the lymphoid tissues, the connective tissues, the kidney, the urinary bladder and other tissues where MMP-9 is expressed. In some embodiments, the promoter is a neuron-selective promoter or a neuron-specific promoter. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Combination Therapies

In some embodiments, the method of treating, ameliorating, and/or preventing the disease and/or disorder contemplated herein includes administering to the subject the effective amount of at least one compound and/or composition contemplated within the disclosure.

In some embodiments, the subject is further administered at least one additional agent that treats, ameliorates, and/or prevents the disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating neurodegenerative conditions, and/or at least one additional agent that treats one or more diseases or disorders contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 g to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of neurodegenerative conditions in a patient.

Formulations may be employed in admixtures with conventional excipients, i.e., pharmaceutically acceptable organic or inorganic carrier substances suitable for intracranially, intrathecal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the neurodegenerative condition in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Related Information and Overview

The progressive transformation of muscle, tendon, ligament, aponeurosis, and fascia into extraskeletal bone tissue is a fundamental signature of fibrodysplasia ossificans progressiva (FOP; MIM #135100), the most catastrophic form of extraskeletal bone formation in humans. Individuals with FOP appear normal at birth except for characteristic malformations of the great toes that are present in all classically affected individuals. During the first decade of life, episodic soft tissue swellings (or flare-ups) arise in the neck and back and undergo pathological metamorphosis into mature heterotopic bone through an endochondral pathway. Minor trauma such as intramuscular immunizations, mandibular blocks for dental work, muscle over-exertion, blunt muscle trauma, bumps, bruises, falls, or influenza-like viral illnesses can trigger flare-ups of FOP that lead to progressive heterotopic ossification (HO).

Most patients are immobilized by the age 30 and require lifelong assistance with activities of daily living. The median estimated lifespan is 56 years; death often results from complications of thoracic insufficiency syndrome. Definitive treatments are not yet available for FOP, and there is an unmet need for effective therapies. Standard-of-care medical management is currently supportive.

Heterozygous missense mutations in activin receptor A type I/Activin-like kinase 2 (ACVR1/ALK2), a bone morphogenetic protein (BMP) type I receptor, have been identified in all individuals with sporadic or familial FOP. ACVR1 mutations cause loss of autoinhibition of ACVR1 and renders it susceptible to dysregulated BMP pathway signaling. Importantly, activin A, a member of the transforming growth factor-β (TGF-0) family of molecules that antagonizes BMP signaling in a wild-type (WT) ACVR1 background, specifically enhances BMP pathway signaling within cells harboring the ACVR1^{R206H} mutation and drives heterotopic bone formation in FOP.

Loss of autoinhibition of the mutant receptor is necessary for the myriad developmental features of FOP, but does not appear sufficient to induce the episodic flare-ups that lead to disabling post-natal HO. FOP flare-ups strongly implicate an underlying inflammatory trigger.

The instant study identified the Patient-R at age of 22 years who has classic developmental features of FOP and the canonical ACVR1 (R206H) mutation but has extreme resilience to the post-natal features of FOP now at age 35 years including the lack of disabling flare-ups and severe progressive HO. The presence of classic developmental anomalies of FOP and the paradoxical paucity of post-natal features of FOP led to the hypothesis that patient-R lacked an inflammatory trigger for flare-up initiation and subsequent heterotopic ossification. Biomarker analysis and genetic studies in patient-R revealed decreased basal activity of matrix metalloproteinase-9 (MMP-9) and compound heterozygosity for MMP-9. Those observations prompted studies in FOP mice that revealed that even partial inhibition of MMP-9 activity by genetic, pharmacologic or biologic means potently inhibited HO and unveiled an unexpected molecular target in FOP and possibly more common forms of HO.

### Example 2: Case Report

An asymptomatic Patient-R was seen in the FOP clinic at age of 22 years. He was the product of a full-term pregnancy and a caesarian section delivery. Short malformed toes and thumbs with absence of interphalangeal joints were noted at birth. He had routine childhood immunizations without flare-ups. At three years of age, he suffered a venomous Loxosceles spider bite on his left thigh. There was no dermonecrosis, but he developed a one-centimeter bony nodule in his left quadriceps which did not affect movement. He enjoyed an active childhood with contact sports and experienced no flare-ups or loss of movement. There is no family history of skeletal malformations or HO.

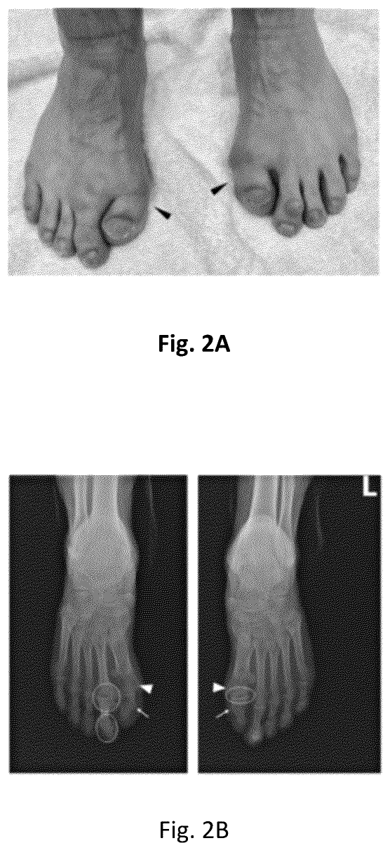

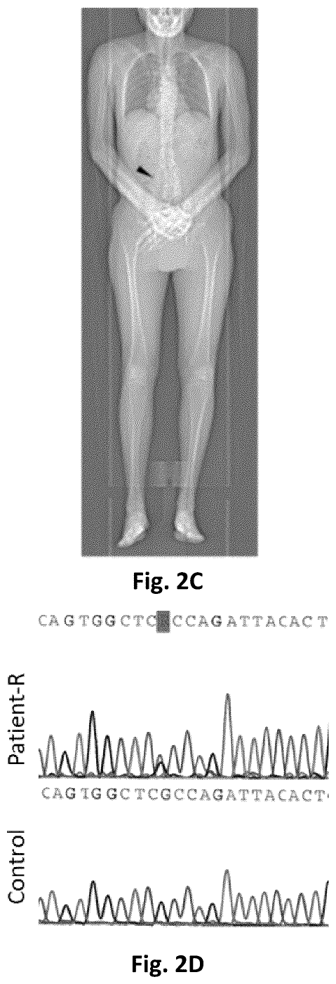

At 21 years of age, he developed a firm nodule on the left side of his neck after multiple episodes of vomiting from a suspected viral gastroenteritis. Excisional biopsy of the nodule revealed a fibroproliferative lesion. A two-centimeter, non-restricting ribbon of HO developed at the operative site. A skeletal survey revealed classic developmental findings of FOP including malformed toes and thumbs, ankylosis of several facet joints of the subaxial cervical vertebrae, short broad femoral necks, dysplasia of the hips, proximal medial tibial osteochondromas and a small amount of asymptomatic HO in the lower lumbar spine (FIG. 2A). Genetic testing confirmed the classic FOP mutation (ACVR1 c.617G>A; R206H). There was no evidence of chimerism.

Physical examination revealed a healthy 22-year-old male with absence of interphalangeal joints of the thumbs and great toes, bilateral proximal medial tibial osteochondromas, and normal range of motion of all other joints of the axial and appendicular skeleton, with the exception of slight decreased motion of the cervical spine and hips. His Cumulative Analogue Joint Involvement Scale (CAJIS) score, a validated tool for assessing FOP disease burden was 2/30 (age related range: 12-22; median: 18). Patient-R participated in a sponsored natural history study of classically-affected FOP patients in which baseline whole body computed tomography (WBCT) was performed to evaluate the total body burden of HO. The WBCT volume of HO for patient-R was 47,000 mm³ (mean for 13 subjects between 25-35 years of age: 434,000 mm³).

Patient-R has the mildest form of classic FOP reported to date for a patient of his age with one minor post-traumatic flare-up of FOP and approximately 90 percent less heterotopic bone compared to age-matched controls. The presence of classic developmental features of FOP (FIG. 2A) suggested that the mutant receptor was active during embryogenesis. However, the paucity of postnatal inflammatory flare-ups of FOP and subsequent HO as well as the extraordinary preservation of mobility in an adult with FOP suggested that he might lack an inflammatory trigger for flare-ups and HO.

Efforts were made to identify the factor(s) that contribute to the resilience to postnatal flare-ups and HO in this unique now 35-year-old individual with FOP (patient-R) and to determine whether this factor(s) were causally related to HO in accurate mouse models of FOP. The identification of such a factor(s) could unveil previously unknown protective mechanisms in FOP and herald new therapeutic strategies for preventing and treating the inexorable progression of this disabling condition.

### Example 3: Methods and Materials

### Patient Study Approvals

Patient-R and unaffected control samples were obtained with informed consent. Research protocols were approved by the Investigational Review Board of the Perelman School of Medicine of the University of Pennsylvania. All human biopsies were obtained prior to a diagnosis of FOP since tissue trauma in FOP frequently induces episodes of heterotopic ossification.

### Animal Study Approvals

All animal procedures were reviewed and approved by the Institutional Animal Care and Use Committee at University of Pennsylvania.

### Venous Blood Collections

Venous blood samples were collected from FOP patients and their family members as part of routine clinical care visits in the Department of Orthopaedic Surgery at the Perelman School of Medicine of the University of Pennsylvania. Blood was collected in 10 ml K2 EDTA tubes (BD cat #366643) for adults and children more than two years old or 4 ml K2 EDTA tubes (BD cat #367861) for children under two years of age. Samples were maintained at room temperature until processing 2-24 hours later.

Peripheral blood mononuclear cells (PBMCs) were obtained as follows: Buffy coat was diluted 1 to 2 with phosphate buffered saline (PBS) and was slowly layered on top of an equal volume of Ficoll-Paque, centrifuged at 400g for 30 minutes to achieve separation of PBMCs from erythrocytes. The mononuclear cell layer was carefully collected and rinsed twice with PBS. PBMCs were stored at −80° C.

### Preparation of Venous Blood Samples

Plasma was separated by one of two methods, depending on whether PBMCs were isolated from the samples for other reasons. Undiluted blood was layered over Ficoll-Paque (GE Healthcare Cat #17-1440-02) in one to two 15 ml conical tubes, and centrifuged at room temperature for 20 min at 800×g with no brake. Plasma was collected from the top layer (leaving behind PBMCs, red blood cells, and Ficoll) and then transferred to a new 15 ml tube. The plasma was centrifuged for 10 min at 1400×g with brake to remove residual cells, and the supernatant was aliquoted into 1.5 ml cryovials. Samples were stored at or below −80° C. Alternatively, undiluted blood was centrifuged in the original collection tube at 1400×g for 20 minutes at room temperature. Plasma was transferred to a new 15 ml tube and centrifuged for 10 min at 1400×g to remove residual cells. The resulting supernatant was aliquoted into 1.5 ml cryovials and stored at or below −80° C.

One cryovial for each chosen sample was thawed on ice, mixed briefly by flicking the tube, and then 500 μl aliquots were distributed into 1.5 ml screw-top microfuge tubes which had been pre-labeled and chilled. These were flash frozen in a dry-ice/ethanol bath and then organized into freezer boxes. These were stored at −80° C. and transported by overnight shipment on dry ice to Myriad Rules Based Medicine (RBM; Salt Lake City, UT) for multiplex analysis.

### Multiplex Luminex Analysis

Multiplex Luminex analysis was performed by Myriad RBM (Pignolo et al., J Bone Miner Res., 2021). All samples were stored at less than −70° C. until tested. Samples were thawed at room temperature, vortexed, spun at 3700×g for 5 min for clarification and transferred to a master microtiter plate. Using automated pipetting, an aliquot of each sample was added to individual microsphere multiplexes of the selected Multi Analyte Profile and blocker. This mixture was thoroughly mixed and incubated at room temperature for 1 hour. Multiplexed cocktails of biotinylated reporter antibodies were added robotically and after thorough mixing, incubated for an additional hour at room temperature. Multiplexes were labeled using an excess of streptavidin-phycoerythrin solution, thoroughly mixed and incubated for 1 hour at room temperature. The volume of each multiplexed reaction was reduced by vacuum filtration and washed 3 times. After the final wash, the volume was increased by addition of buffer for analysis using a Luminex instrument and the resulting data interpreted using proprietary software developed by Myriad RBM.

For each multiplex, both calibrators and controls were included on each microtiter plate. Eight-point calibrators to form a standard curve were run in the first and last column of each plate and controls at 3 concentration levels were run in duplicate. Standard curve, control, and sample QC were performed to ensure proper assay performance. Study sample values for each of the analytes were determined using 4 and 5 parameter logistics, with weighted and non-weighted curve fitting algorithms included in the data analysis package.

### Cell Culture

PBMCs were defrosted, suspended in AIM-V medium and plated at a density of 2.5×10⁶ cells/ml in 24-well plates and incubated at 37° C. in a 5% C02 atmosphere. PBMC were incubated with 20 ng/ml of TNF-α for 72 hours. Cells after lysis with RIPA Lysis and Extraction buffer (ThermoFisher Scientific, Grand Island, NY, USA; #89901) with 1× proteinase inhibitor cocktail (ThermoFisher Scientific, Grand Island, NY, USA; #78429) were collected for analysis also as cell culture supernatants.

### Clinical Determination of Flare-Up Status

Forty patient samples, with age- and sex-matched controls to the extent possible, were stratified on the basis of flare-up status at the time of sample collection (Pignolo et al., J Bone Miner Res, 2021). The present study measured the levels of 113 analytes in plasma samples from four subject groups: unaffected individuals and individuals with FOP by flare-up status (active, remote, quiescent). Flare-up status was arbitrarily defined by the time from appearance of symptoms and signs clinically determined to be consistent with the last episodic exacerbation at the time of sample collection. Flare-up status was defined as following: (1) an active flare-up is a current/ongoing clinical flare-up at the time of sample collection; (2) a flare-up is remote when has occurred within 1-2 years of sample collection; and (3) quiescent status occurs when the last flare-up occurred more than 2 years before sample collection.

### Protein-Protein Interaction (PPI) Mapping

PPI mapping was performed by IBM-Watson for Drug Discovery (WDD) and Ingenuity software and was used to generate relationship networks from biomarker predictions. Biological relationship network extraction was applied to proteins significantly associated with FOP genotype and flare-up status based on previously described methods for analysis of pathway interactions between differentially expressed proteins. The confidence was set to >95%, and captured links were supported by at least two published documents.

FOP Mice (Skeletal Muscle Injury with Cardiotoxin-Minocycline and Doxycycline Treatment)

A conditional-on knock-in mouse model Acvr1^{[R206H]FlEx} (Acvr1^{R206H/+}) was used to generate tamoxifen-inducible global R206H mutant allele expression after recombination by Cre recombinase (Hatsell et al., Sci Transl Med. 2015 Sep. 2; 7(303):303ra137). Acvr1^{R206H/+} and MMP-9^{-/-} (The Jackson Laboratory, Bar Harbor, ME, USA; Stock #007084) mice were crossed with CreERT2 mice (The Jackson Laboratory, Bar Harbor, ME, USA; Stock #008463) to generate Acvr1^{R206H/+};CreERT2^{-/+}, Acvr1^{R206H/+};CreERT2^{-/+};MMP-9^{-/+} and Acvr1^{R206H/+};CreERT2^{-/+};MMP-9^{-/-} mice.

Mice were injected intraperitoneally 5 times over 2 weeks (starting at 4 weeks of age) with tamoxifen in corn oil (100 mg/kg body weight, 10 mg/ml stock solution, Sigma, St. Louis, M0, USA; #T5648) to induce inversion of the R206H mutant allele.

Quadriceps muscles (at 7 weeks of age) were injured by injecting 50 μL of 10 μM cardiotoxin from Naja mossambica (Sigma-Aldrich, St. Louis, MO, USA; #C9759).

Mice were daily treated intraperitoneally with the minocycline 100 mg/kg (Cayman Chemical, Ann Arbor, Michigan, USA; #14454) or appropriate control in pharmaceutical grade saline 3 days prior to the induction of HO and for 14 days after induction of HO.

Acvr1^{Q207D/+} mice were a gift from Dr. Yuji Mishina (University of Michigan). To induce expression of Q207D followed by HO, AV-Cre (University of Pennsylvania Vector Core; 1×10¹¹ particles per mouse) was injected together with 50 μL of 10 μM cardiotoxin into the popliteal fossa of Acvr11^{Q207D/+} mice at 3-4 weeks of age.

The experimental endpoint for in vivo studies to analyze the appearance of heterotopic ossification was determined to be 2 weeks, based on the fact that mice in a control group had developed HO by this time after muscle injury with cardiotoxin.

### MMP-9 Monoclonal Antibodies Related Methods and Materials

Mice were treated with antibodies against MMP-9 (GS-622703, Gilead Sciences, Foster City, California, USA) at 50 mg/kg on day −3 and then 15 mg/kg on day 0 (the induction of HO) and day 3 or appropriate control.

### Quantification of MMP9

Total MMP9 protein (92 kDa pro- and 82 kDa active forms) was determined by sandwich ELISA (Human MMP-9 Quantikine ELISA Kit, DMP900 and Mouse Total MMP-9 Quantikine ELISA Kit, MMPT90, R&D Systems, Minneapolis, MN, USA) in heparin treated platelet-poor plasma.

### Zymography

Gelatin zymography was performed using an equal amount of cell supernatants and cells after lysis (10 ug) for measurement of MMP9 with Novex Zymogram Plus Gels according manufacturer's instructions (Invitrogen, Carlsbad, CA, USA, ZY00100BOX).

The gels were scanned, the images were inverted and densitometry levels were determined by using the ImageJ software (imagej dot nih dot gov/ij/). The generated profile yielded peaks corresponding to each band (pro-MMP-9 and active-MMP-9).

### Immunocyto/Histochemistry

Tissue samples were fixed in 4% paraformaldehyde for 24 hours and decalcified using 10% EDTA for 7 days, snap frozen or embedded in paraffin, and sectioned serially at 8 μm. Deparaffinized sections and frozen sections were treated for antigen retrieval with 10 mM sodium-citrate buffer (pH 6.0) at 95° C. for 20 min.

For immunofluorescence staining, sections were blocked using Background Buster (Innovex Biosciences, Richmond, CA, USA; NB306), incubated with primary antibodies overnight at 4° C., then with appropriate secondary antibodies conjugated with Alexa 594 and Dapi mounting medium. Images were acquired on Nikon microscope with NIS software.

For immunohistochemistry endogenous peroxidase activity was quenched. Sections were blocked, incubated with primary antibodies overnight at 4° C., then with appropriate host horseradish peroxidase (HRP) secondary antibody, DAB detection (SuperPicture Polymer 879263; Thermo Fisher Scientific), and hematoxylin/eosin counter stain.

Results were compared to negative controls processed without primary antibody. Primary antibodies used were as follows: MMP-9 (Millipore, Sigma, Burlington, MA, USA; AB19016; 1:100 dilution).

CRISPR gene editing was used to insert 59C>T and 493G>A into MMP-9 in THP-1 human leukemia monocytic cell line according to protocol from ThermoFisher Scientific using TrueCut HiFi Cas9 Protein.

### Micro-Computerized Tomography

Micro computerized tomography (μCT) was performed on hind limbs from mice obtained 14 days after cardiotoxin or adenovirus-Cre/cardiotoxin injection using a Scanco VivaCT 40 device (Bruettisellen, Switzerland) to determine the volume of heterotopic bone and obtain a two-dimensional image of the medial sagittal plane of each limb. Scanning was performed using a source voltage of 55 kV, a source current of 142 A, and an isotropic voxel size of 10.5 m. Bone was differentiated from “non-bone” by an upper threshold of 1000 Hounsfield units and a lower threshold of 150 Hounsfield units.

### Structural Modeling

Sequence-based predicative algorithms, molecular modeling, and molecular dynamics simulations were used to test the potential for the Ala20Val and the Asp165Asn variants to alter the structure and activity of MMP9.

### Statistical Analyses

All experiments were performed with at least three technical and biological replicates, with sample sizes indicated in the figure legends. GraphPad Prism 8.0 software (San Diego, CA, USA) was used for statistical analysis. Data were checked for normality using the D'Agostino & Pearson normality test. Parametric data were analyzed using an appropriate Student's t test when two groups were being compared, or a one-way (or two-way) analysis of variance was used when more than two groups were compared (or with at least 2 independent factors), respectively, followed by a post hoc Tukey's test to compare two groups. Nonparametric data were analyzed with a Mann-Whitney U test when two groups were being compared or a Kruskal-Wallis one-way analysis when more than two groups were compared. *P<0.05, **P<0.01, and ***P<0.001 were considered significant. Data are represented as mean (+/−) standard error of the mean (SEM).

### Example 4: FOP Patient-R, Who Exhibits Congenital Features of FOP but Lacks Post-Natal Progressive Heterotopic Ossification, had Suppressed Inflammatory Biomarkers

Patient-R was a 35-year-old man with the classical Acvr1R206H mutation. Referring to FIGS. 2A-2C, Patient-R had classic developmental findings of FOP, including malformed toes and thumbs (FIGS. 2A-2B), ankylosis of several facet joints of the subaxial cervical vertebrae, short broad femoral neck, dysplasia of the hips, proximal medial tibial osteochondromas, and small amount of asymptomatic HO in the lower lumbar spine. The presence of classic big toe malformation in FOP suggested that the mutant Acvr1 receptor in Patient-R was active during early development.

However, Patient-R showed resilience to the post-natal features of FOP. For example, the disabling flare-ups and severe progressive HO found in essentially all other FOP patients are absent in Patient-R (FIG. 2C).

To elucidate the mechanism behind the resilience to the post-natal features of FOP in Patient-R, the present study measured the level of inflammatory biomarkers in the plasma of Patient-R, as well as the plasma of other FOP patients and control subjects. 113 plasma proteins, including chemokines and cytokines (Custom Human MAP panel), were measured by quantitative multiplexed immunoassays (Myriad RBM) and analyzed in the blood collected from the FOP patients and the control subjects. The analysis showed that Patient-R has paucity of post-natal inflammatory markers compared to others with FOP. As shown in Table 1, Biomarkers of inflammation in Patient-R were significantly suppressed compared to those with quiescent FOP, as the statistical probabilities of observing the observed low amounts for these biomarkers (as calculated in p-values) are exceedingly small. Notably, the p-value for the plasma MMP-9 level in Patient-R was calculated to be lower than 6.8×10⁻⁵.

TABLE 1
Plasma Analytes of Inflammation in Patient-RR Were
Suppressed Compared to Those with Quiescent FOP*.

| Biomarkers | p-value (<) |
|---|---|
| CRP | 0.03 |
| FRTN | 0.03 |
| IgA | 0.05 |
| IL-1ra | 0.02 |
| IL-1RI | 0.05 |
| IL-18 | 0.02 |
| KLK-7 | 0.01 |
| MIP-1β | 0.001 |
| MIF | 0.01 |
| MMP-2 | 0.04 |
| MMP-9 | 6.8 × 10⁻⁵ |
| TN-C | 0.02 |
| TIMP-1 | 3.0 × 10⁻⁴ |
| TNFR | 0.02 |

Plasma analytes of inflammation were significantly suppressed in Patient-R (two independent samples from blood draws more than a year apart) compared to those with quiescent FOP* (N = 11 individuals; one sample per individual). P-values are noted for each sample. CRP = C-reactive protein; FRTN = Ferritin; IgA = Immunoglobulin A; IL-1ra = Interleukin-1 receptor antagonist; IL-1R1 = Interleukin-1 receptor type 1; IL-18 = Interleukin-18; KLK-7 = Kallekrein-7; MIP-1β = Macrophage inflammatory protein-1beta; MIF = Macrophage inhibitory factor; MMP-2 = Matrix metalloproteinase-2; MMP-9 = Matrix metalloproteinase-9; TN-C = Tenascin-C; TIMP-1 = Tissue inhibitor of metalloproteinases-1; TNFR = Tumor necrosis factor-2. The following plasma analytes of inflammation were not significantly different between Patient-RR and those with quiescent FOP: Adiponectin; Compliment-3; Interleukin-1 receptor type 2; Matrix metalloproteinase-3; Prostasin; T-cell-specific RANTES; Thymus expressed chemokine; Tissue inhibitor of metalloproteinases-3. The following plasma analytes of inflammation were too low to be detected in all samples (Patient-R and Quiescent group): B lymphocyte chemoattractant; Granulocyte-macrophage colony-stimulating factor; Interferon gamma; Interleukin-1 alpha; Interleukin-1 beta; Interleukin-2, 3, 4, 5, 6, 7, 8, 10, 15, 17, 23; Macrophage inflammatory proein-1 alpha; Matrix metalloproteinases 1, 7, 10; Monocyte chemotactic protein-1, 3; Transforming growth factor beta-3; Tumor necrosis factor alpha; Tumor necrosis factor beta.
*Quiescent FOP group is defined as last flare-up occurring at least 2 years prior to plasma sample collection

### Example 5: Patient-R had Low Plasma MMP-9 Levels and was a Compound Heterozygote for Polymorphic Variants in MMP-9

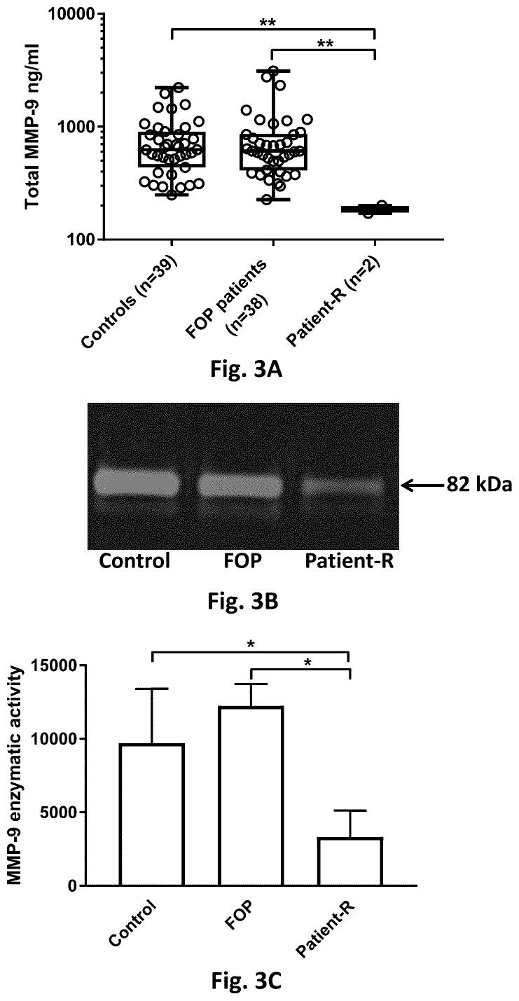

As shown in FIGS. 3A-3C, Patient-R has low plasma MMP-9 levels compared to other FOP patients. Referring to FIGS. 3A-3C, the present study compared a commercially-available multiplex set of 113 plasma-soluble analytes in patient-R to the same analytes in 40 FOP patients with the classic ACVR1^{R206H} mutation as well as 40 age- and gender-matched controls. It was found that total MMP-9 was significantly lower (p<0.0002) in patient-R compared to others with the classic ACVR1R^{206H} mutation as well as in age- and gender-matched controls (Pignolo et al., J Bone Miner Res, 2021).

Whole exome sequencing was performed in patient-R and 19 additional FOP patients with a classic phenotype using DNA isolated from blood or saliva, in accordance with the UCSF Biospecimens and Skeletal Tissues for Rare and Orphan Genetics (BSTROnG) and University of Pennsylvania cohorts and sequenced as described in the Methods section. Ingenuity Variant Analysis (IVA, Qiagen) confirmed that all 20 patients with FOP were heterozygous for ACVR1^{R206H}, and no additional ACVR1 mutations were identified in any of the subjects. Approximately 80,000 total variants were identified for each FOP patient as compared to the human reference genome, UCSC Browser GrCh38.

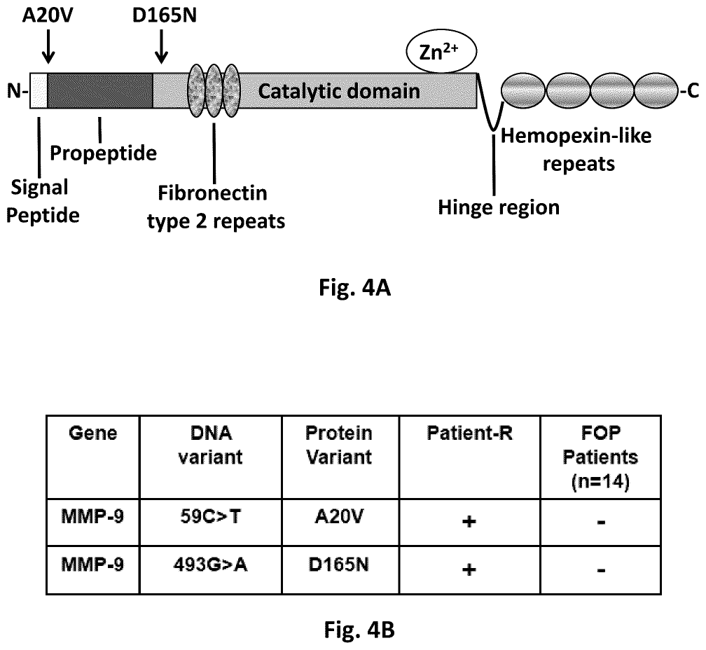

Patient-R had several unique variants compared to the 19 classic FOP patients. All of the unique variants were further classified according to pathogenicity using the American College of Medical Genetics (ACMG) Standards and Guidelines. The present study identified two polymorphisms in MMP-9 that were transmitted from two different alleles based on genetic sequencing of MMP-9 from the parents of patient-R (FIGS. 4A-4B). Specifically, one allele of MMP-9 gene in Patient-R has a polymorphism resulting in an A20V protein sequence change (Rs1805088—A20V—Frequency in population for T=0.02201 [https://www.ncbi.nlm.nih.gov/snp/rs1805088 #frequency_tab]), while the other allele of MMP-9 gene in Patient-R has a polymorphism resulting in a D165N protein sequence change (Rs8125581—D165N—Frequency in population for A=0.000360 [https://www.ncbi.nlm.nih.gov/snp/rs8125581 #frequency_tab]).

### Example 6: Protein Structure Modeling of MMP-9 Polymorphisms in Patient-R Suggested Less Functionality than Normal Alleles

In silico studies of the present study suggest that the A20V variant of MMP-9 likely disrupts signal peptide cleavage, interfere with translation on the rough endoplasmic reticulum (ER), impair folding inside the ER or disrupt tethering of MMP-9 to membranes of the secretory pathway.

D165N polymorphism is located within the catalytic domain of MMP-9, is predicted by multiple computational approaches to be highly deleterious to MMP9 structure and function and to result in a non-functional variant (Bhatnager et al., Computational Biology and Chemistry, Volume 77, December 2018, Pages 97-108). The D165N variant of MMP-9 corresponds to the metal binding region of the catalytic domain of MMP9 and the stability of MMP9 depends on the interaction of the peptide with the calcium ion (Bhatnager, 2018). Thus, the D165N variant is likely ineffective in inhibiting enzymatic activity as it cannot form the requisite ion pair with His118 as in wildtype MMP-9. Importantly, the D165N variant of MMP-9 is predicted to be highly unstable, which would diminish folding efficiency in the endoplasmic reticulum, a site of quality control regulating the trafficking of properly folded secretory proteins. Since the ion pair stabilizes a loop just several residues C-terminal of the site of MMP-9 cleavage and activation, the loop of the variant D165N MMP-9 protein might not adopt a conformation optimal for proteolysis by the activating enzyme plasminogen (Gong et al., J Clin Invest 118:3012-3024, 2008).

While the D165N single nucleotide polymorphism (SNP) discovered in patient-R is rare and non-pathogenic in the general population, this variant was predicted to be protective in patients with aortic aneurysm. Furthermore, the specific enzymatic activity (total activity/secreted protein amount) was studied for the D165N MMP-9 variant and was found to be significantly lower than that for wild-type MMP-9, suggesting a substantial effect of the amino acid substitution on the enzymatic activity of MMP-9 in addition to a decrease in secreted protein. The MMP-9 variants (A20V and D165N) detected in patient-R present a structural basis for loss-of-function of secreted MMP-9 and have implications for suppression of HO in FOP.

### Example 7: MMP-9 is Expressed in Early Lesional Tissues in Subjects Suffering from FOP

In certain embodiments, biopsies of early lesional tissue from classically affected FOP human patients (i.e., human FOP patients who have the Acvr1 R206H mutation and display the post-natal symptoms of FOP) show positive MMP-9 staining in the inflammatory cells.

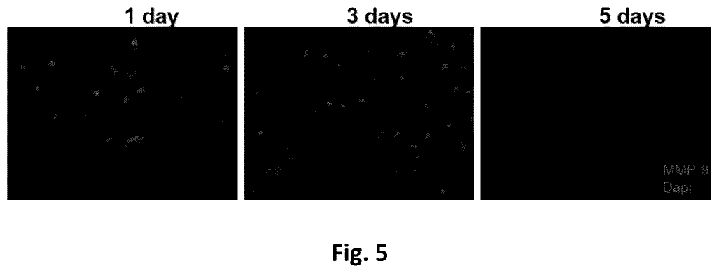

Similar results were found in FOP model mice. Referring to FIG. 5, images of lesional tissue of FOP model mice (Acvr1^{R206H/+};CreERT2^{-/+}) after cardiotoxin injury show that MMP-9 was expressed early on in the inflammatory cells in the lesion area. Furthermore, the expression of MMP-9 gradually reduces in the lesional tissue from day 1 to day 5.

### Example 8: Down-Regulation of MMP-9 Level or Activity Reduces Heterotopic Ossification in Mouse FOP Models

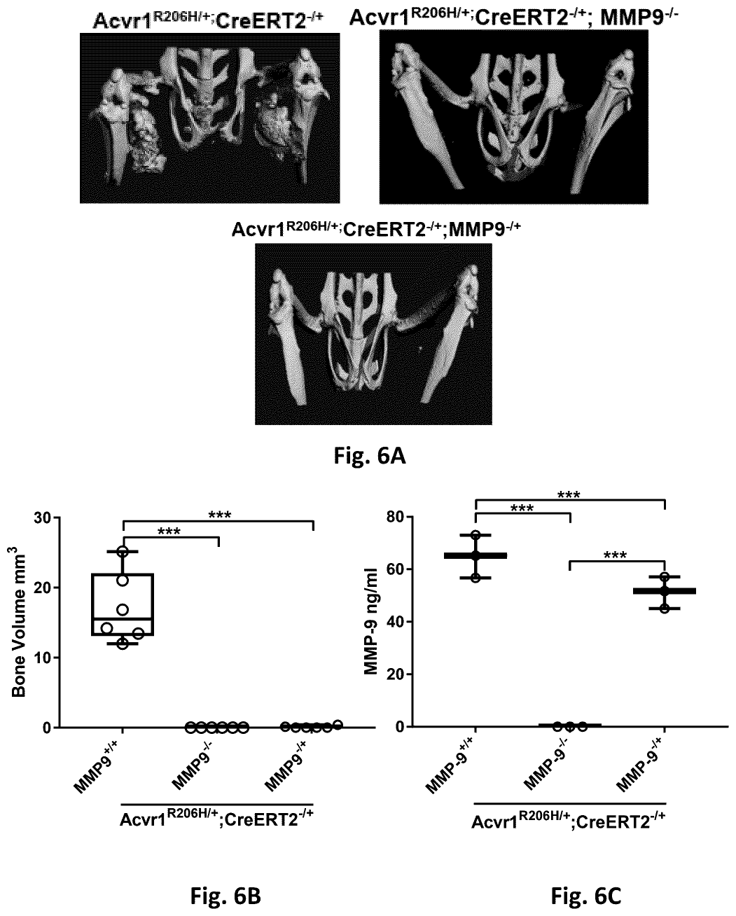

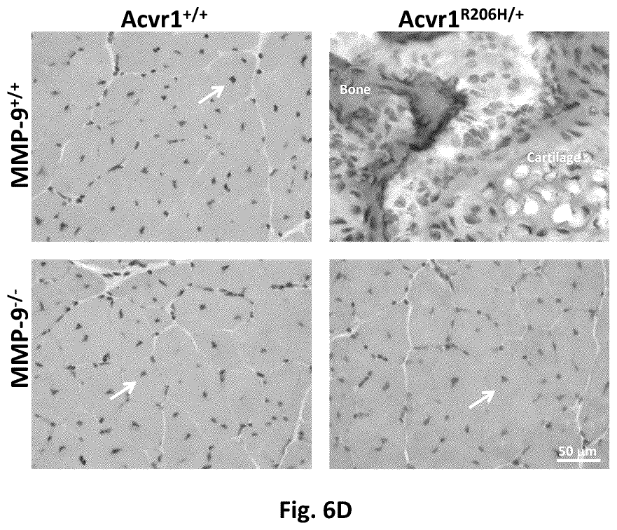

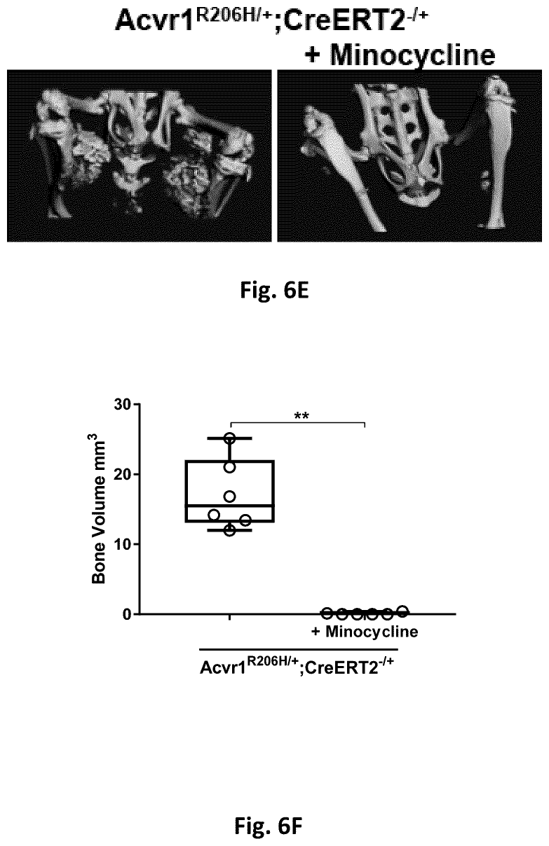

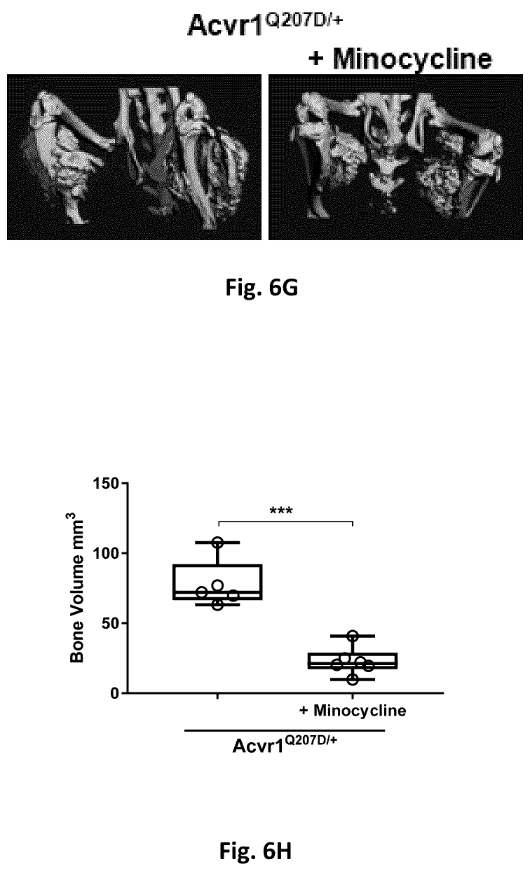

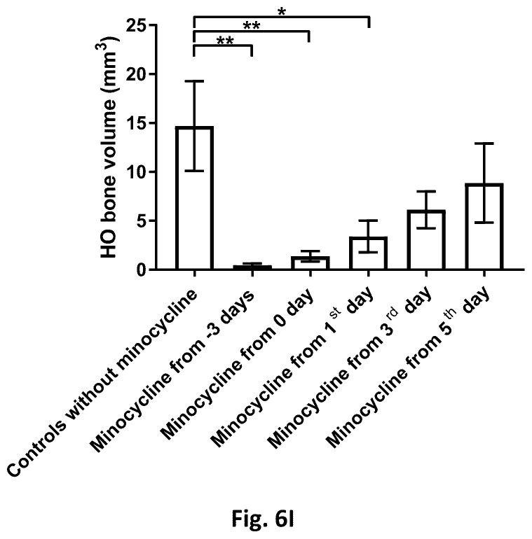

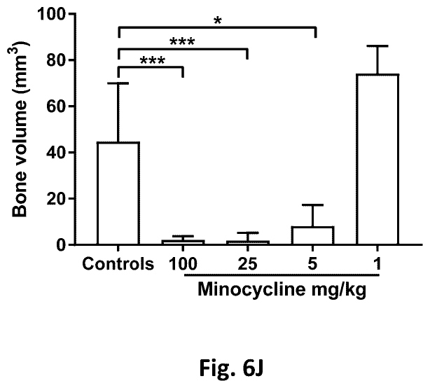

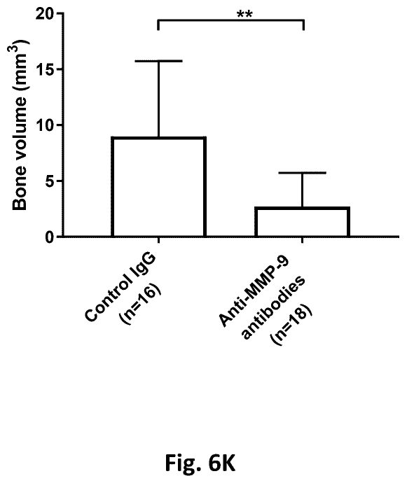

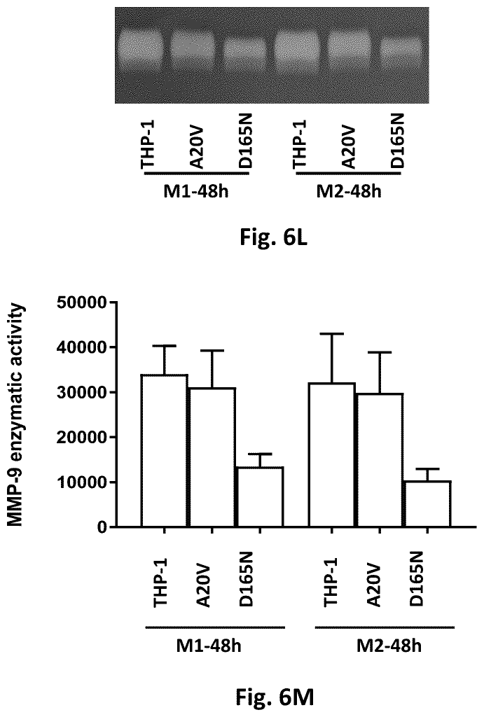

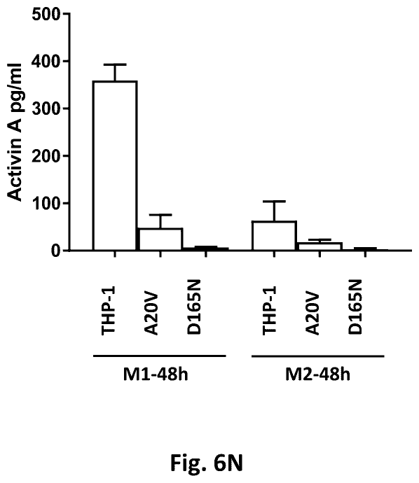

Referring to FIGS. 6A-6C, soft tissue injuries were induced in Acvr1^{R206H/+};CreERT2^{-/+} mice (FOP model mice having the classical R206H mutation in one Acvr1 allele), Acvr1^{R206H/+};CreERT2^{-/+};MMP-9^{-/+} mice (FOP model mice having only one allele of MMP-9 gene) or Acvr1^{R206H/+};CreERT2^{-/+};MMP-9^{-/-} mice (FOP model mice having both alleles of MMP-9 gene deleted) with cardiotoxin, and levels of heterotopic ossification (HO) in response to the injury were detected by microCT and quantitated.

As shown in FIG. 6C, MMP9^{-/+};Acvr1^{R206H/+};CreERT2^{-/+} and MMP9^{-/-};Acvr1^{R206H/+};CreERT2^{-/+} mice produce significantly less MMP-9 than Acvr1^{R206H/+};CreERT2^{-/+} control mice.

As visualized in FIG. 6A and quantitated in FIG. 6B, Acvr1^{R206H/+};CreERT2^{-/+} mice showed high levels of HO while Acvr1^{R206H/+};CreERT2^{-/+};MMP-9^{-/-} mice showed significantly lower levels of HO. Interestingly, Acvr1^{R206H/+};CreERT2^{-/+};MMP-9^{-/+} mice showed levels of HO significantly lower than those of Acvr1^{R206H/+};CreERT2^{-/+} mice and comparable to those of Acvr1^{R206H/+};CreERT2^{-/+};MMP-9^{-/-} mice, indicating that even partial reductions of MMP-9 expression level and activity were sufficient to reduce the injury induced HO in response to soft tissue injuries in the FOP model mice.

Since small molecule compounds that inhibit MMP-9 activities are available, the study tested the effects of one such MMP-9 inhibitor, minocycline, in present the FOP model mice.

Minocycline is a broad-spectrum tetracycline antibiotic, and is used to treat many different bacterial infections, such as urinary tract infections, respiratory infections, skin infections, severe acne, gonorrhea, Rocky Mountain spotted fever, chlamydia, and others.

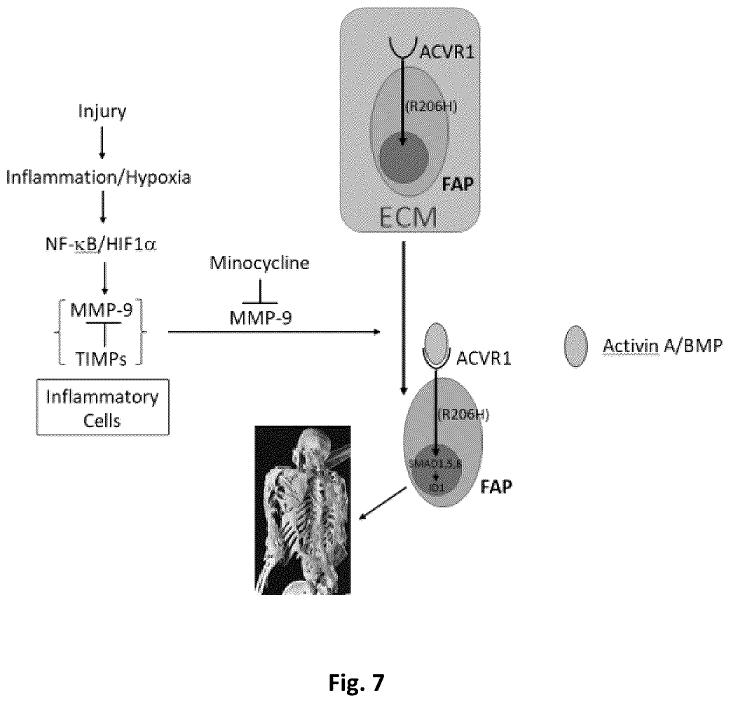

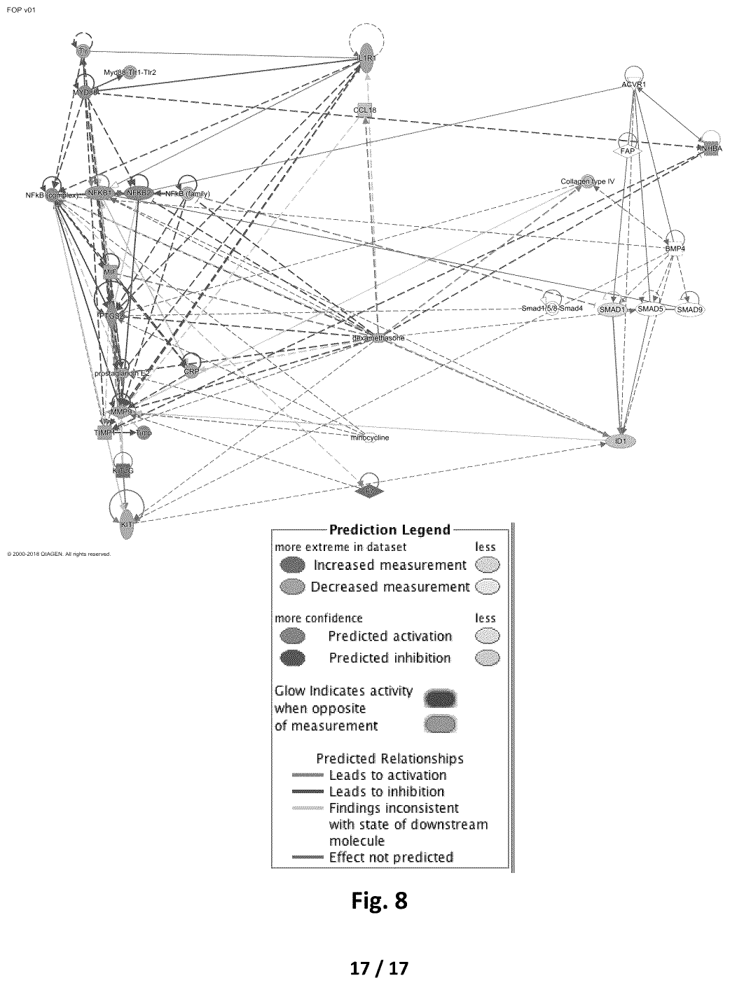

Referring to FIGS. 7D-7F, Acvr1^{R206H/+};CreERT2^{-/+} FOP mouse model, as well as another mice FOP mouse model Acvr1^{Q207D/+} (Fukuda et al, Genesis. 2006 April;44(4):159-67) were used to study the effects of minocycline in heterotopic ossification caused by cardiotoxin induced soft tissue injuries.

Referring to FIGS. 6D-6F, minocycline almost fully eliminated soft tissue injury induced HO in the Acvr1^{R206H/+};CreERT2^{-/+} FOP mice. Referring to FIGS. 6G-6H, minocycline significantly reduced level of soft tissue injury induced HO in Acvr1^{Q207D/+} FOP mice, as well.

Referring to FIG. 6I, Minocycline protects against HO when the compound was administered before or at the time of lesional activation. The data indicates that MMP-9 acts to induce HO during the early inflammatory stage.

The present study then sought to down-regulate MMP-9 using an anti-MMP-9 monoclonal antibody (Marshall et al. PLoS One. 2015 May 11; 10(5):e0127063). Referring to FIG. 6K, in Acvr1^{R206H/+};CreERT2^{-/+} FOP mice, the administration of the antibody significantly reduced the heterotopic ossification in the FOP mouse model.

### Example 9: Selected Discussion

The present study shows that MMP-9 plays a critical role in the pathogenesis of HO in FOP. The presence of classic developmental findings of FOP (osteochondromas in the proximal tibias, congenital cervical spine fusion, hallux valgus malformations and shortened thumbs) along with an extreme paucity of post-natal flare-ups and HO in patient-R suggested that FOP was active during embryogenesis but relatively inactive post-natally, possibly due to a lack of an inflammatory trigger for HO. The findings of significantly decreased MMP-9 reserve and activity in patient-R along with putative protective polymorphisms for the MMP-9 gene drew attention to that factor which was further validated as contributory to the phenotype of HO inhibition by in vivo studies in FOP mice. Taken together, these results support that MMP-9 mediates HO by regulating the inflammatory response to tissue metamorphosis.

MMP-9 (or gelatinase B) belongs to a multi-gene family of more than 20 matrix metalloproteinases that are conserved throughout the animal kingdom and that process or degrade numerous pericellular substrates. MMP-9 plays an essential and multi-faceted role as a modulator of inflammation in remodeling the extracellular matrix; activating, deactivating and/or modifying pro-inflammatory cytokines and other signaling molecules and orchestrating the migration of stem cells and tissue progenitor cells in a large spectrum of physiologic and pathological processes including embryonic development, skeletal morphogenesis, fracture repair, inflammation, wound healing, angiogenesis, heart disease, arthritis and cancer. Specifically, MMP-9 triggers the degradation and remodeling of the extracellular matrix, the release and activation of growth factors, the remodeling of the stem cell niche and the recruitment and migration of inflammatory cells and stem cells.

MMP-9 is expressed in neutrophils, macrophages, and mast cells, is regulated by inflammatory cytokines, is a modulator of inflammation and innate immunity, integrates multiple immunoregulatory pathways and promotes disruption of biochemical and physical barriers to T-cell trafficking. MMP-9 is also expressed at elevated levels in ischemic skeletal muscle which exists in early FOP flare-ups.

MMP-9 is also a signal inducer of endochondral ossification. MMP-9 regulates chondrogenic and osteogenic cell differentiation during early stages of fracture repair and is expressed throughout the entire process of fracture repair. MMP-9^{-/-} mice display abnormal fracture healing and non-unions.

The role of MMP-9, if any, in the pathogenesis of FOP has not been investigated. The clinical, biochemical, and genetic findings in patient-R by the present study indicate that even a partial reduction in MMP-9 levels might protect against disabling flare-ups and HO in the presence of the classic ACVR1^{R206H} mutation, as haploinsufficiency of MMP-9 is protective against HO in FOP mice.

The in vivo studies in genetically accurate mouse models of trauma-induced FOP confirm this hypothesis and demonstrate that MMP-9 plays a pivotal role in the genesis of HO and that genetic, pharmacologic, and biologic reductions in MMP-9 levels protect against the induction of HO in mouse models of FOP. Taken together, these data indicate that pharmacologic reduction in tissue levels of MMP-9 in patients with FOP can protect against HO.

The present study noted that the tetracycline class of pharmaceuticals. Tetracycline has been used therapeutically for nearly 40 years as an antibiotic against both gram-positive and gram-negative bacteria. However, in addition to its antibiotic properties, minocycline and doxycycline which are second-generation semi-synthetic tetracyclines are potent inhibitors of MMP-9 at sub-antimicrobial doses.

In summary, the present study has identified MMP-9 as an important modulator of HO in FOP and demonstrated that systematic study of a single resilient individual can unveil unexpected mechanisms of disease that may lead to novel treatment strategies.

### ENUMERATED EMBODIMENTS

In some aspects, the present invention is directed to the following non-limiting embodiments:

Embodiment 1: A method of treating, ameliorating, and/or preventing fibrodysplasia ossificans progressiva (FOP) in a subject in need thereof, the method comprising down-regulating matrix metalloproteinase 9 (MMP-9) level and/or activity in the subject.

Embodiment 2: The method of Embodiment 1, wherein down-regulating the MMP-9 level and/or activity in the subject comprises administering to the subject an effective amount of:
a small molecule MMP-9 inhibitor,
a protein MMP-9 inhibitor,
a nucleic acid (and/or an expression vector expressing the nucleic acid) that downregulates MMP-9 by RNA interference,
a ribozyme (and/or a vector expressing the ribozyme) that downregulates MMP-9,
an expression vector comprising an expression cassette, wherein the expression cassette expresses CRISPR components that downregulate MMP-9 by CRISPR knockout and/or CRISPR knockdown, and
a trans-dominant negative mutant protein of MMP-9, and/or an expression vector that expresses the trans-dominant negative mutant protein of MMP-9.

Embodiment 3: The method of any one of Embodiments 1-2, wherein the subject has a mutant ACVR1 gene.

Embodiment 4: The method of Embodiment 3, wherein the mutant ACVR1 gene encodes a constitutively active ACVR1 polypeptide.

Embodiment 5: The method of Embodiment 4, wherein the ACVR1 polypeptide comprises at least one mutation selected from the group consisting of L196P, P197-F198 del ins L, R202I, R206H, Q207E, F246Y, R258S, R258G, G325A, G328E, G328W, G328R, G356D, R375P, and K400E.

Embodiment 6: The method of any one of Embodiments 2-5, wherein the small molecule MMP-9 inhibitor is selected from the group consisting of doxycycline, incyclinide, and minocycline, or a salt or solvate thereof.

Embodiment 7: The method of any one of Embodiments 2-5, wherein the protein MMP-9 inhibitor is an anti-MMP-9 antibody or an antigen binding fragment thereof.

Embodiment 8: The method of any one of Embodiments 1-7, further comprising surgically removing an ossified tissue from the subject.

Embodiment 9: The method of Embodiment 8, wherein surgically removing step is performed after the MMP-9 level or activity in the subject is down-regulated.

Embodiment 10: The method of any one of Embodiments 1-9, wherein the subject is a human.

Embodiment 11: A kit for treating, ameliorating and/or preventing fibrodysplasia ossificans progressiva (FOP) in a subject in need thereof, the method comprising down-regulating matrix metalloproteinase 9 (MMP-9) level and/or activity in the subject, comprising:
a compound for down-regulating matrix metalloproteinase 9 (MMP-9) level and/or activity in the subject; and
an instruction for administering an effective amount of the compound to the subject.

Embodiment 12: The kit of Embodiment 11, wherein the compound comprises at least one selected from the group consisting of:
a small molecule MMP-9 inhibitor,
a protein MMP-9 inhibitor,
a nucleic acid (and/or an expression vector expressing the nucleic acid) that downregulates MMP-9 by RNA interference,
a ribozyme (and/or a vector expressing the ribozyme) that downregulates MMP-9,
an expression vector comprising an expression cassette, wherein the expression cassette expresses CRISPR components that downregulate MMP-9 by CRISPR knockout and/or CRISPR knockdown, and
a trans-dominant negative mutant protein of MMP-9, and/or an expression vector that expresses the trans-dominant negative mutant protein of MMP-9.

Embodiment 13: The kit of any one of Embodiments 11-12, wherein the subject has a mutant ACVR1 gene.

Embodiment 14: The kit of Embodiment 13, wherein the mutant ACVR1 gene encodes a constitutively active ACVR1 polypeptide.

Embodiment 15: The kit of Embodiment 14, wherein the ACVR1 polypeptide comprises at least one mutation selected from the group consisting of L196P, P197-F198 del ins L, R202I, R206H, Q207E, F246Y, R258S, R258G, G325A, G328E, G328W, G328R, G356D, R375P, and K400E.

Embodiment 16: The kit of any one of Embodiments 11-15, wherein the small molecule MMP-9 inhibitor is selected from the group consisting of doxycycline, incyclinide, and minocycline.

Embodiment 17: The kit of any one of Embodiments 11-15, wherein the protein MMP-9 inhibitor is an antibody against MMP-9 or an antigen binding fragment thereof.

Embodiment 18: The kit of any one of Embodiments 11-17, wherein the manual further comprises instructions for surgically removing an ossified tissue from the subject.

Embodiment 19: The kit of Embodiment 18, wherein the instruction further comprises instructions to perform the surgically removing after the level or the activity of MMP-9 in the subject is down-regulated.

Embodiment 20: The kit of any one of Embodiments 11-19, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of treating, ameliorating, or preventing fibrodysplasia ossificans progressiva (FOP) in a subject in need thereof, the method comprising down-regulating matrix metalloproteinase 9 (MMP-9) level or activity in the subject.

2. The method of claim 1, wherein down-regulating the MMP-9 level or activity in the subject comprises administering to the subject an effective amount of:
a small molecule MMP-9 inhibitor,
a protein MMP-9 inhibitor,
a nucleic acid that downregulates MMP-9 by RNA interference, or an expression vector expressing the nucleic acid that downregulates MMP-9 by RNA interference,
a ribozyme that downregulates MMP-9, or a vector expressing the ribozyme that downregulates MMP-9,
an expression vector comprising an expression cassette, wherein the expression cassette expresses CRISPR components that downregulate MMP-9 by CRISPR knockout or CRISPR knockdown, and
a trans-dominant negative mutant protein of MMP-9, or an expression vector that expresses the trans-dominant negative mutant protein of MMP-9.

3. The method of claim 1, wherein the subject has a mutant ACVR1 gene.

4. The method of claim 3, wherein the mutant ACVR1 gene encodes a constitutively active ACVR1 polypeptide.

5. The method of claim 4, wherein the ACVR1 polypeptide comprises at least one mutation selected from the group consisting of L196P, P197-F198 del ins L, R202I, R206H, Q207E, F246Y, R258S, R258G, G325A, G328E, G328W, G328R, G356D, R375P, and K400E.

6. The method of claim 2, wherein the small molecule MMP-9 inhibitor is selected from the group consisting of doxycycline, incyclinide, and minocycline, or a salt or solvate thereof.

7. The method of claim 2, wherein the protein MMP-9 inhibitor is an anti-MMP-9 antibody or an antigen binding fragment thereof.

8. The method of claim 1, further comprising surgically removing ossified tissue from the subject.

9. The method of claim 8, wherein the surgically removing step is performed after the MMP-9 level or activity in the subject is down-regulated.

10. The method of claim 1, wherein the subject is a human.

11. A kit for treating, ameliorating or preventing fibrodysplasia ossificans progressiva (FOP) in a subject in need thereof, comprising:
a compound for down-regulating matrix metalloproteinase 9 (MMP-9) level or activity in the subject; and
an instruction for administering an effective amount of the compound to the subject.

12. The kit of claim 11, wherein the compound comprises at least one selected from the group consisting of:
a small molecule MMP-9 inhibitor,
a protein MMP-9 inhibitor,
a nucleic acid that downregulates MMP-9 by RNA interference, or an expression vector expressing the nucleic acid that downregulates MMP-9 by RNA interference,
a ribozyme that downregulates MMP-9, or a vector expressing the ribozyme that downregulates MMP-9,
an expression vector comprising an expression cassette, wherein the expression cassette expresses CRISPR components that downregulate MMP-9 by CRISPR knockout or CRISPR knockdown, and
a trans-dominant negative mutant protein of MMP-9, or an expression vector that expresses the trans-dominant negative mutant protein of MMP-9.

13. The kit of claim 11, wherein the subject has a mutant ACVR1 gene.

14. The kit of claim 13, wherein the mutant ACVR1 gene encodes a constitutively active ACVR1 polypeptide.

15. The kit of claim 14, wherein the ACVR1 polypeptide comprises at least one mutation selected from the group consisting of L196P, P197-F198 del ins L, R202I, R206H, Q207E, F246Y, R258S, R258G, G325A, G328E, G328W, G328R, G356D, R375P, and K400E.

16. The kit of claim 12, wherein the small molecule MMP-9 inhibitor is selected from the group consisting of doxycycline, incyclinide, and minocycline.

17. The kit of claim 12, wherein the protein MMP-9 inhibitor is an antibody against MMP-9 or an antigen binding fragment thereof.

18. The kit of claim 11, wherein the instruction further instructions for surgically removing an ossified tissue from the subject.

19. The kit of claim 18, wherein the instruction further comprises instructions to perform the surgically removing after the level or the activity of MMP-9 in the subject is down-regulated.

20. The kit of claim 11, wherein the subject is a human.
